(19) United States
(12) Patent Application Publication
(10) Pub. No.: US 2003/0130194 A1
(43) Pub. Date: Jul. 10, 2003

(54) Method and device for increasing and/or decreasing the concentration of immunomodulatory-active substances in substance mixtures

(76) Inventors: Jens Altrichter, Kavelstorf (DE); Jens Freytag, Rostock (DE); Steffen Mitzner, Rostock (DE); Jan Stange, Rostock (DE)

Correspondence Address: Paul & Paul, 2900 Two Thousand Market Street, Philadelphia, PA, 19103, US

(21) Appl. No.: 10/348,522

(22) Filed: Jan. 20, 2003

Related U.S. Application Data
(63) Continuation of application No. 10/181,291, filed on Jul. 15, 2002.
Application No. 10/181,291 is a 371 of international application No. PCT/DE00/00130, filed on Jan. 14, 2000.
(63) Continuation-in-part of application No. 09/352,987, filed on Jul. 14, 1999, now Pat. No. 6,509,147.

(30) Foreign Application Priority Data
Jul. 16, 1998 (DE) 19831873.1

(51) Int. Cl. A61K 48/00; A61K 45/00; A61K 38/17

(52) U.S. Cl. 514/12; 514/44; 424/93.7

(57) ABSTRACT

The invention relates to a method for increasing and/or decreasing the concentration of immunomodulatory-active substances in substance mixtures which contain potentially immunomodulatory-active substances, and corresponding device for carrying out said method

The invention relates to a method for increasing and/or decreasing the concentration of immunomodulatory-active substances in substance mixtures which contain potentially immunomodulatory-active substances, and a corresponding device for carrying out said method.

### STATE OF THE ART

The immune system and its modulation have the last few years increasingly been the focus of fundamental and related research not only in the fields of biotechnology, cosmetics and medicine, but also of food and environmental technology. The reasons for this lie on the one hand especially in the occurrence of new infections, and the spread of old ones, but also the increasing occurrence of substances with immunomodulatory potential, such as e.g. substances in foodstuffs, cosmetics or the environment which inadequately alter the immune system in its function, thereby causing e.g. allergies or autoimmune diseases.

Infections

The most serious form of an infection, in which the pathogens have spread throughout the whole body, is known as sepsis. During the course of a sepsis, a number of changes occur in the activity of the components of the immune system, which, should critical levels be exceeded, ends in the death of the organism. Current knowledge concerning the course and pathogenesis of a sepsis essentially stems from investigations into the interactions between Gram-negative bacteria and the human organism (Chest 1992; 101; 1644-1655). The chief agents in the introduction of a sepsis cascade are correspondingly represented by the bacterial endotoxins, a group of lipopolysaccharides from the cell wall of Gram-negative bacteria (Reviews Infect Dis. 1983; 5; 733-747). Endotoxins, probably the most potent fever-producing substances (pyrogens) of all, activate especially monocytes and endothelium cells. By the release of mediators/formation of adhesion molecules, the immune system is activated, and leukocyte sticking prepared. This leads to migration of the leukocytes into tissue with a high chemotaxin content (place of local inflammation). If the local cause can be removed, then the inflammation process is stopped. If, in the longer tem, this results in intermittent or continuous excessive flooding of bacteria, endotoxins or other antigenic cell products into the blood, the beneficial defence reaction of monocytes and endothelium cells changes into an autoaggressive process with very serious circulatory dysregulations, secondary organ failures, coagulation disturbances (DIC) etc.: a sepsis (with positive pathogens detected) and/or a systemic inflammatory response syndrome (SIRS, no pathogens detectable) develop and lead to the death of the patient in up to 30% of cases of simple sepsis, and up to 90% of cases of septic shock (Sepsis, an interdisciplinary challenge. Berlin, Heidelberg, N.Y.; Springer Verlag, 1989).

Gram-positive pathogens as a cause of sepsis have in the last few years increasingly been the focus of research. Numerous works are concerned with the rising incidence of Gram-positive sepsis in the last decade. Statistics show that already 30 to 40% of all cases of sepsis can be ascribed to Gram-positive pathogens (Am J Med. 1991; 91 (suppl 3B): 72-89). The treatment of bacterial sepsis in intensive medicine centres is in addition made more difficult due to the increasing resistance to antibiotics. At present sepsis is regarded as presenting a multi-phase clinical picture, in which the phase of flooding with germs is followed by a so-called hyperinflammation phase with an excessive outpouring of pro-inflammatory cytokines, such as e.g. tumour-necrosis factor alpha (TNF-alpha) or some interleukins (e.g. interleukin-1 and interleukin-6). Later, due to negative re-coupling mechanisms, there is an excessive change in favour of the anti-inflammatory cytokines (transforming growth factor beta=TGF beta, interleukin-4, interleukin-10, interleukin-13) thus leading to the so-called immunoparalysis phase, which finally results in the death of the patient (Internist 1997; 38; 541-552). However, so far a clear definition of the individual phases based on concrete paraclinical values has not yet been produced.

So far it has only been possible to attempt the causal treatment of bacterial sepsis. Hopes are founded, in addition to antibiotic therapy, on the application of anti-inflammatory substances, which are currently being investigated for their effects on Gram-negative sepsis (Nature, 1990; 348; 550-552, FASEB J. 1991; 5: 338-343). Some such studies have already been concluded and have so far produced somewhat disappointing results. Thus, neither antibodies directed against endotoxin-lipid A (N. Engl. J. Med. 1991; 324; 429436, JAMA 1991; 266; 1097-1102) nor anti-cytokine therapies against tumour-necrosis-factor alpha (Crit Care Med. 1993; 21; 318-327, JAMA 1995; 273; 934-941) or interleukin-1 (JAMA 1994; 271; 1836-1842) contribute to a reduction in overall mortality. However, patients with very high cytokine levels at least seem to benefit to some extent (Crit Care Med. 1993; 21; 318-327, Crit Care Med. 1996; 24; 733-742). The studies are however characterized by a high level of heterogeneity of the patient groups and hitherto inadequate division of the sepsis development into clinical stages.

A new approach is the use of pro-inflammatory cytokines (interferon gamma) in the immunoparalysis phase, which appears promising in initial non-randomised studies, but so far still shows a mortality rate of more than 30% (Nature Medicine 1997; 3; 678-681). Immunomodulatory approaches for which applications have been made under patent law include in particular the administration of individual immunomodulatory substances, such as, e.g. some cytokine antagonists described above (WO 94/06431 A1, U.S. Pat. No. 5,585,486, U.S. Pat. No. 5,585,357, U.S. Pat. No. 5,565,430, U.S. Pat. No. 5,552,400). Mistletoe lectin (DE 4221836), fosfomycin (JP 09183730 A), macrocyclic substances (U.S. Pat. No. 5,527,907, U.S. Pat. No. 5,541,189, U.S. Pat. No. 5,541,193, U.S. Pat. No. 5,561,139, U.S. Pat. No. 5,561,140) or bacterial extracts (WO 89/09607, EP 0 363 491). None of these approaches was able to demonstrate decisive therapeutic advantages in sepsis therapy.

An interesting approach is the use of granulocyte or macrophage-stimulating factors (G-CSF, GM-CSF). It has been possible to favourably influence the development of serious sepses by the administration of such factors (Blood 1999; 93: 425-439; Curr Opin Hematol 1999; 6: 176-183: J Infect Dis 1999; 179: p. 342-352; Arch Pediatr Adolesc Med 1999; 153: 984-988). The favourable clinical effect is best achieved by increasing the degree of differentiation and the specific functionality of white blood cells (granulocytes and macrophages).

Allergies

The term “allergies” is used to describe overreactions of the immune system caused by previous sensitisation to a substance not normally occurring naturally in the body. They are generally triggered by small chemical groups (haptens) which are linked to a larger chemical structure (the hapten carrier, the whole complex is referred to as so-called complete allergen). A distinction is made between different types of allergic reactions. In particular type I is brought about by an increased formation of immunoglobulin E type antibodies which are directed against the hapten/allergen. The binding and cross-linking of the immunoglobulin E molecules at the surface receptors by the allergens leads to activation of the immune cells (predominantly mass cells and basophilic granulocytes) with subsequent formation and release of a series of immunomodulatory molecules (e.g. histamine, prostaglandin, leukotrienes and cytokines such as TNF-alpha or various interleukins) (Bundschuh, G, Lexikon der Immunologie, 2^{nd} Edition, Medical Service, Munich 1992). Existing immunomodulatory therapies used in allergy treatment concentrate above all on blocking the release of the immunomodulating substances by immunosuppressives (e.g. glucocorticoids) and/or inhibiting the binding of the substances to the target cells (e.g. histamine- or leukotriene-receptor antagonists). Another form of therapy involves administering small quantities of the allergen and slowly increasing the dose to achieve desensitisation (Bundschuh, G, Lexikon der Immunologie, 2^{nd} Edition, Medical Service, Munich 1992).

Extracorporeal Immunomodulation

Extracorporeal attempts to detoxify the blood have to date involved the use of chemical adsorbers to deplete germ components (e.g. DEAE cellulose, polyethylenimine or PMX fibre as endotoxin adsorbers (Mitzner et al. Artificial Organs 1992, 17, 775-781, Samtleben et al. Artificial Organs 1998, 22, 4346; Tani et al. Artifical Organs 1998, 22, 1038-1044).

The immunomodulatory approaches known hitherto have predominantly been carried out by in-vivo administration of substances. These substances include e.g. antibiotics, which prevent germs from multiplying and thus eliminating the germs as immunomodulatory agents. More recent attempts at immunomodulation have been carried out with antibodies directed against cytokines and/or other protein preparations binding the cytokines and have resulted in no, or only slight, therapeutic benefits. (Internist 1997; 38: 541-552). Endotoxin adsorbers have to date exhibited good depletions in vitro. In extracorporeal therapy of serious infections, endotoxin adsorbers have not to date been used in controlled, randomised studies. A non-randomised study with PMX fibres however also found a reduction in the mortality rate to only 46%, which is still an unsatisfactorily high figure (Tani et al. Artificial Organs 1998, 2, 1038-1044).

In conditions of hyperinflammation or immunoparalysis, there is an increase in blood plasma concentrations of a number of immunomodulating substances such as e.g. cytokines, germ components and germ products, some of which considerably disturb the defence of infections. The elimination or addition of an individual cytotoxine has so far produced no convincing therapeutic success. Rather the useful removal of too-high concentrations of individual substances and the substitution of other substances which are present in too-low concentrations are at the same time required to stabilise the immune system when it has gone off course. The complexity of the problem of changing concentrations of the immunomodulating substances requires on the one hand sensitive measurement, and on the other hand the ability to rapidly substitute the appropriate substances. Due solely to the delay in determining the cytokine concentration, substitution therapies in the form of injections/infusions will in future only conditionally be able to contribute to the solution.

The object of the present invention is therefore to provide a method which is improved with regard to prior art, and a device for altering the concentration of immunomodulatory-active substances in a substance mixture or a solution.

According to the invention this object is achieved by a method of the type mentioned at the start, in which the substance mixture or the solution is introduced into a bioreactor and brought into contact with cells therein, said cells being selected such that they comprise receptors for at least one species of a group G1 of immunomodulatory-active substances, whose concentration in the substance mixture is to be decreased, and such that they are capable of adsorbing this species, and/or that the cells produce at least one species from another group G2 of immunomodulatory substances, whose concentration in the substance mixture is to be increased, and such that they are capable of releasing this species into the substance mixture, and the substance mixture is then separated from the cells and removed from the bioreactor.

Furthermore, the object according to the invention is achieved by a device for increasing and/or decreasing the concentration of immunomodulatory-active substances in a substance mixture with a bioreactor with an inlet for introducing the substance mixture and an outlet for removing the substance mixture, living cells being present in the bioreactor in a form such that the substance mixture thereof can be brought into contact with the cells, and with a cell-retaining device, which is designed so that the substance mixture can be separated from the cells and removed free from cells from the reactor, the cells having receptors for at least one species of a group G1 of immunomodulatory-active substances whose concentration in the substance mixture is to be decreased, and which are capable of adsorbing this species and/or that the cells produce at least one species of another group G2 of immunomodulatory-active substances, whose concentration in the substance mixture is to be increased, and which are capable of releasing this species into the substance mixture.

The substance mixture used in the method according to the invention is preferably blood, blood plasma, blood plasma components and/or water individually or in combination. It may possibly also contain proteins, peptides, carbohydrates, lipids, nucleic acids, salts and/or microorganisms individually or in combination.

The use of cells which, with their specific surface receptors, selectively adsorb immunomodulating substances and thus remove them from the substance mixture, but which can on the other hand also form and release the actual immunomodulatory-active molecules underrepresented in the substance mixture, such as, e.g. cytokines, represents a new approach, which involves removing substances which are extracorporieally immunologically active in a positive or negative way under controlled conditions from substance mixtures such as blood or blood plasma of a patient, or adding them thereto. The substance mixture can be the blood or a blood component of a patient with an infection, an allergy or another disease, which is attended by an alteration in the concentrations of immunomodulatory-active substances compared with the healthy state. The substance mixture can again be administered to the patient after the method according to the invention is carried out. However the substance mixture cannot originate from the same patient, but for example from the blood of another individual, such as a unit of stored blood, or it may be a synthetically prepared solution, which is enriched with immunomodulatory-active substances by means of the method according to the invention and then administered to a patient. By the present invention it becomes possible for the first time to carry out a complex “intelligent” biological modification of immunological process and to control this in a targeted way.

The method according to the invention allows the work to be carried out under physiological conditions (temperature, pH values, ion concentrations, buffers etc.) which makes possible considerable advantages in the processing of thermolabile and otherwise sensitive physiological substance mixtures. The cell system according to the invention can be easily monitored for stability of morphological and functional properties. This is of particular significance from the point of view of the maximum possible safety sought, and selectivity of the processes occurring. Also, and precisely for industrial application, the fact that operations taking place can be described precisely is of great significance and indispensable e.g. with regard to questions of licensing and quality standards.

The groups of immunomodulatory-active substances described herein, G1 for adsorbed substances and G2 for released or secreted substances, are not to be understood as having to be different from one another, or at least being unable to contain any identical substances. Rather it is the case that frequently the respective concentration of a substance determines whether this substance has a positive or negative effect from an immunological viewpoint, i.e. whether it is to be adsorbed or released in the substance mixture in question. In the method according to the invention, the same substances can also be contained in both Groups G1 and G2, if the cells used both adsorb and also release these, thus producing a concentration balance. The term “substances” as used within the meaning of the invention also includes living organisms such as bacteria, fungi, yeasts etc., which are immunomodulatory-active.

According to the invention it is advantageous if the groups G1 and/or G2 of immunomodulatory-active substances include cytokines, preferably pro-inflammatory or anti-inflammatory cytokines, soluble cytokine receptors, cytokine receptor antagonists, growth factors, soluble adhesion molecules, components or decomposition products of the coagulation-, fibrinolysis or complement system and reactive oxygen species.

Especially preferably, the groups G1 and/or G2 include the known immunomodulatory-active substances interferon alpha, interferon beta, interferon gamma, interleukin 1, interleukin 2, interleukin 3, interleukin 4, interleukin 5, interleukin 6, interleukin 7, interleukin 8, interleukin 9, interleukin 10, interleukin 11, interleukin 12, interleukin 13, interleukin 14, interleukin 15, interleukin 16, interleukin 17, interleukin 18, tumour-necrosis-factor alpha (TNF-alpha), tumour-necrosis-factor beta (TNF-beta), transforming growth factor beta (TGF-beta), chemokine and chemotaxin. Also especially preferred are endotoxins, preferably lipopolysaccharides, exotoxins, preferably haemolysin A, B, C, D or E, lipoteichoic acid and zymosan. Also suitable are immunocomplexes, comprising at least one antibody, preferably an immunoglobin, or an antibody part and at least one antigen or antigenically active substance, such as a hapten.

According to the invention the groups G1 and/or G2 can also contain components or products of microorganisms, preferably bacteria, viruses, fungi or parasites, or complete microorganisms or allergenically active substances.

For the method according to the invention, it has proved especially advantageous if the cells in the bioreactor are leukocytes, haematopoietic stem cells, cells obtained from haematopoietic stem cells by differentiation, haematocytes, endothelium cells, nerve cells, mucosa cells, epithelium cells or other cells originating from the ectoderm, mesoderm or endoderm, or a combination thereof. Especially suitable are primary cells, immortalised or tumour cells. Although cells of any origin can be used in the method according to the invention, cells of human, animal, plant or microbial origin are particularly suitable.

In an especially preferred embodiment of the invention, the cells in the bioreactor are stimulated before or during introduction and contact with the substance mixture. By using specific pre-stimulated (“trained”) immunocells, a disturbance in the immunological balance can be detected on the one hand by the cells functioning as multi-parametric biosensor and at the same time this disturbance can be completely or partly evened out.

To achieve the highest possible vitality of the cells used, and to influence the metabolic activity, it is expedient to regulate the temperature, gassing, and food supply of the cells in the bioreactor in a known manner. A cell filter or centrifuge is suitable as a cell retention device, with which the substance mixture in the bioreactor is separated after contact with the cells.

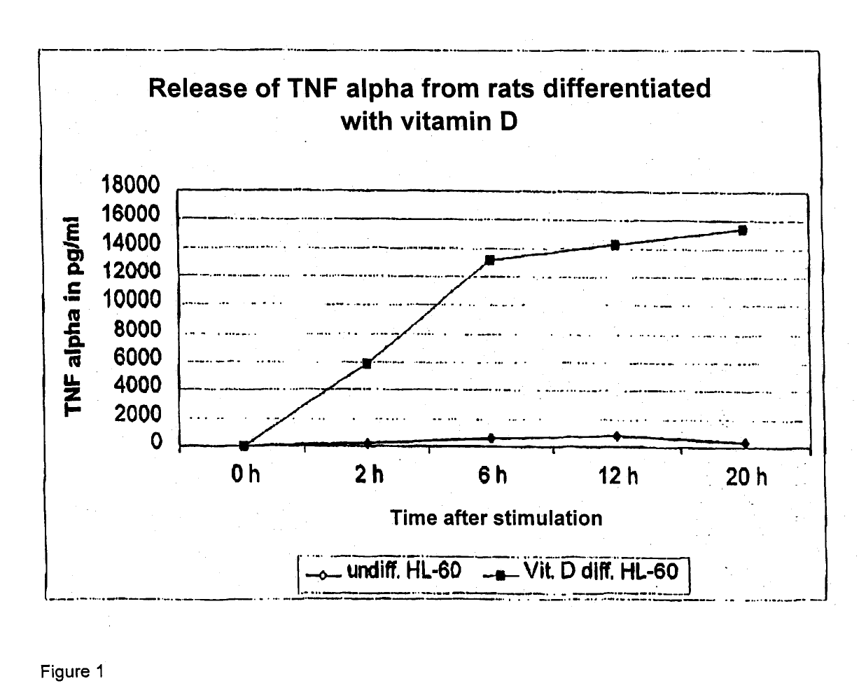

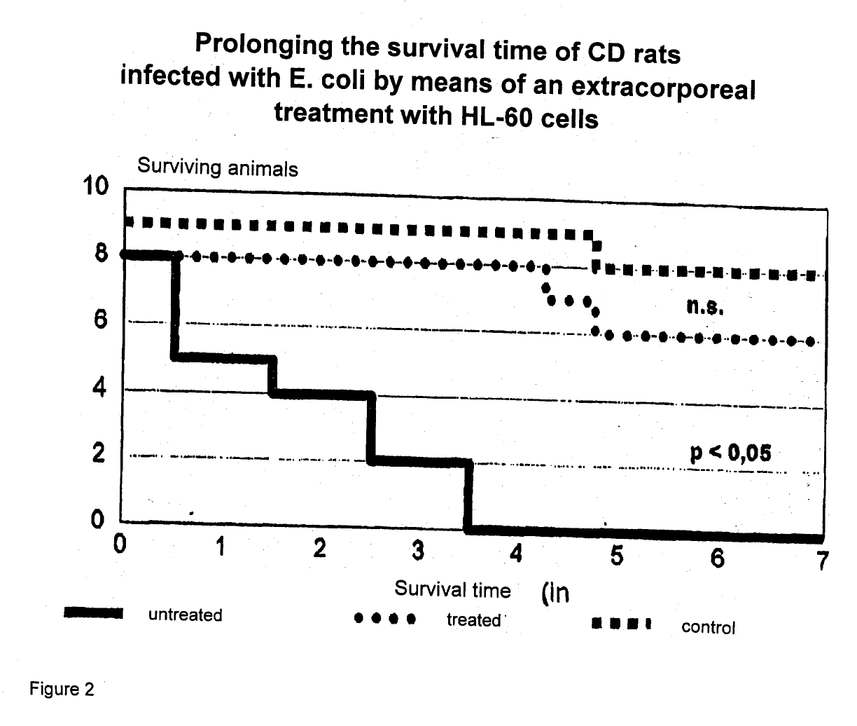

Further advantages, features and embodiments of the present invention are made clear with reference to the following example and the accompanying FIGS. 1 and 2.

### EXAMPLE

Blood from CD rats, which had been treated with Escherichia coli bacteria was separated by filtration or differential centrifugation into corpuscular components, such as blood cells, and the blood plasma separated (plasmapheresis) and the blood plasma or blood plasma components subsequently passed through the bioreactor according to the invention. HL60 cells which had previously been stimulated and differentiated with vitamin D were used as cells in the bioreactor. As is known, via specific receptors, HL60 cells bind cytokines, such as interferon gamma, and growth factors such as granulocyte-colony stimulating factor, as well as other immunomodulating substances. By adsorption of these substances at the corresponding receptors, the cells reduced their concentration in the plasma introduced into the bioreactor, whilst on the other hand releasing actual immunomodulating substances, such as interleukin 6 and tumour-necrosis-factor alpha (TNF-alpha). The concentration of released TNF-alpha in the blood plasma was determined at the moment of introduction into the bioreactor and taken as reference value for the quantity of the TNF-alpha released by the stimulated cells. After 2, 6, 12 and 20 hours in the bioreactor the plasma was separated from the cells and removed from the bioreactor, and in each case the concentration of TNF-alpha in the plasma was determined. As a control, undifferentiated HL60 cells were used.

The results of this experiment are graphically represented in FIG. 1. The concentration of TNF-alpha in the plasma rose steeply in the first 6 hours following stimulation of the HL60 cells, then somewhat less steeply, but steadily.

The bioreactor eluate immunomodulated with the stimulated HL60 cells (20 hours in the bioreactor) was then again brought together with the corpuscular blood components, the rats were reinfused and the survival rate of the rats determined. As a control, the survival rate of infected rats which were not reinfused with treated plasma or blood was determined, as well as the survival rate of non-infected rats, which were reinfused with the eluate. The results of this experiment are graphically represented in FIG. 2.

The rats infected with Escherichia coli bacteria survived considerably longer if the plasma had been treated in the way prescribed according to the invention, indeed almost as long as the non-infected control rats.

1. Method for increasing and/or decreasing the concentration of immunomodulatory-active substances in substance mixtures or solutions which contain potentially immunomodulatory-active substances, wherein
the cells are introduced into a bioreactor and brought into contact with cells therein,
wherein the cells are selected such that they comprise receptors for at least one species of a group G1 of immunomodulatory-active substances, whose concentration in the substance mixture is to be decreased, and such that they are capable of adsorbing this species, and/or such that the cells produce at least one species of another group G2 of immunomodulatory-active substances whose concentration in the substance mixture is to be increased, and such that they are capable of releasing this species into the substance mixture,
the substance mixture is then separated from the cells and removed from the bioreactor.

2. Method according to claim 1, characterized in that the substance mixture contains blood, blood plasma, blood plasma components and/or water individually or in combination and possibly also proteins, peptides, carbohydrates, lipids, nucleic acids, salts and/or microorganisms individually or in combination.

3. Method according to one of claims 1 or 2, characterized in that the groups G1 and/or G2 of immunomodulatory substances contain cytokines, preferably proinflammatory or anti-inflammatory cytokines, soluble cytokine receptors, cytokine receptor antagonists, growth factors, soluble adhesion molecules, components or decomposition products of the coagulation-, fibrinolysis or complement system and reactive oxygen species.

4. Method according to one of claims 1 to 3, characterized in that the groups G1 and/or G2 of immunomodulatory substances contain interferon alpha, interferon beta, interferon gamma, interleukin 1, interleukin 2, interleukin 3, interleukin 4, interleukin 5, interleukin 6, interleukin 7, interleukin 8, interleukin 9, interleukin 10, interleukin 11, interleukin 12, interleukin 13, interleukin 14, interleukin 15, interleukin 16, interleukin 17, interleukin 18, tumour-necrosis-factor alpha (TNF-alpha), tumour-necrosis-factor beta (TNF-beta), transforming growth factor beta (TGF-beta), chemokine and chemotaxin.

5. Method according to one of claims 1 to 4, characterized in that the groups G1 and/or G2 contain components or products of microorganisms, preferably bacteria, viruses, fungi or parasites, or complete microorganisms or allergenically active substances.

6. Method according to one of claims 1 to 5, characterized in that the groups G1 and/or G2 contain endotoxins, preferably lipopolysaccharides, exotoxins, preferably haemolysin A, B, C, D or E, lipoteichoic acid and zymosan.

7. Method according to one of claims 1 to 6, characterized in that the groups G1 and/or G2 contain immunocomplexes, comprising at least one antibody, preferably an immunoglobin, or an antibody part and at least one antigen or antigenically active substance, preferably a hapten.

8. Method according to one of claims 1 to 7, characterized in that the cells in the bioreactor are leukocytes, haematopoietic stem cells, cells obtained from haematopoietic stem cells by differentiation, haematocytes, endothelium cells, nerve cells, mucosa cells, epithelium cells or other cells originating from the ectoderm, mesoderm or endoderm, or a combination thereof.

9. Method according to one of claims 1 to 8, characterized in that the cells in the bioreactor are primary cells, immortalised or tumour cells.

10. Method according to one of claims 1 to 9, characterized in that the cells in the bioreactor are cells of human, animal, plant or microbial origin.

11. Method according to one of claims 1 to 10, characterized in that the cells in the bioreactor are stimulated before or during introduction and contact with the substance mixture.

12. Method according to one of claims 1 to 11, characterized in that the temperature, gassing, and food supply of the cells in the bioreactor are regulated.

13. Method according to one of claims 1 to 12, characterized in that after contact with the cells, the substance mixture is separated from the cells by means of a cell retaining device, preferably a cell filter or centrifuge.

14. Device for increasing and/or decreasing the concentration of immunomodulatory-active substances in a substance mixture with a bioreactor with an inlet for introducing the substance mixture and an outlet for removing the substance mixture, living cells being present in the bioreactor in a form such that the substance mixture thereof can be brought into contact with the cells, and with a cell-retaining device, which is designed so that the substance mixture can be separated from the cells and removed from the reactor free from cells, characterized in that the cells have receptors for at least one species of a group G1 of immunomodulatory-active substances whose concentration in the substance mixture is to be decreased, and which are capable of adsorbing this species and/or that the cells produce at least one species of another group G2 of immunomodulatory-active substances, whose concentration in the substance mixture is to be increased, and which are capable of releasing this species into the substance mixture.

15. Device according to claim 14, characterized in that the substance mixture contains, blood, blood plasma, blood plasma components and/or water individually or in combination and possibly also proteins, peptides, carbohydrates, lipids, nucleic acids, salts and/or microorganisms individually or in combination.

16. Device according to one of claims 14 or 15, characterized in that the groups G1 and/or G2 of immunomodulatory substances contain cytokines, preferably proinflammatory or anti-inflammatory cytokines, soluble cytokine receptors, cytokine receptor antagonists, growth factors, soluble adhesion molecules, components or decomposition products of the coagulation-, fibrinolysis or complement system and reactive oxygen species.

17. Device according to one of claims 14 to 16 characterized in that the groups G1 and/or G2 of immunomodulatory substances contain interferon alpha, interferon beta, interferon gamma, interleukin 1, interleukin 2, interleukin 3, interleukin 4, interleukin 5, interleukin 6, interleukin 7, interleukin 8, interleukin 9, interleukin 10, interleukin 11, interleukin 12, interleukin 13, interleukin 14, interleukin 15, interleukin 16, interleukin 17, interleukin 18, tumour-necrosis-factor alpha (TNF-alpha), tumour-necrosis-factor beta (TNF-beta), transforming growth factor beta (TGF-beta), chemokine and chemotaxin.

18. Device according to one of claims 14 to 17 characterized in that the groups G1 and/or G2 contain components or products of microorganisms, preferably bacteria, viruses, fungi or parasites, or complete microorganisms or allergenically active substances.

19. Device according to one of claims 14 to 18 characterized in that the groups G1 and/or G2 contain endotoxins, preferably lipopolysaccharides, exotoxins, preferably haemolysin A, B, C, D or E, lipoteichoic acid and zymosan.

20. Device according to one of claims 14 to 19 characterized in that the groups G1 and/or G2 contain immunocomplexes, comprising at least one antibody, preferably an immunoglobin, or an antibody part and at least one antigen or antigenically active substance, preferably a hapten.

21. Device according to one of claims 14 to 20 characterized in that the cells in the bioreactor are leukocytes, haematopoietic stem cells, cells obtained from haematopoietic stem cells by differentiation, haematocytes, endothelium cells, nerve cells, mucosa cells, epithelium cells or other cells originating from the ectoderm, mesoderm or endoderm, or a combination thereof.

22. Device according to one of claims 14 to 21 characterized in that the cells in the bioreactor are primary cells, immortalised or tumour cells.

23. Device according to one of claims 14 to 22, characterized in that the cells in the bioreactor are cells of human, animal, plant or microbial origin.

24. Device according to one of claims 14 to 23, characterized in that the cells in the bioreactor are stimulated before or during introduction and contact with the substance mixture.

25. Device according to one of claims 14 to 24, characterized in that the bioreactor is designed so that the temperature, gassing, and food supply of the cells can be regulated.

26. Device according to any one of claims 14 to 25, characterized in that the cell retaining device is a cell filter or centrifuge.
